SEQUENCE LISTING (35 sequences)

SEQ ID NO: 1 - protein, 477 aa, Escherichia coli
MGSLTNNDNLHIFLVCFIGQGVVNPMLRLGKAFASKGLLVTLSAPEIVGTEIRKANNLNDDQPIKVGSGMIRFEFFDDGWESVNGSKPFDVWVYINHLDQTGRQKLPIMLKKHEETGTPVSCLILNPLVPWVADVADSLQIPCATLWVQSCASFSAYYHYHHGLVPFPTESEPEIDVQLPGMPLLKYDEVPDYLHPRTPYPFFGTNILGQFKNLSKNFCILMDTFYELEHEIIDNMCKLCPIKPIGPLFKIPKDPSSNGITGNFMKVDDCKEWLDSRPTSTVVYVSVGSVVYLKQEQVTEMAYGILNSEVSFLWVLRPPSKRIGTEPHVLPEEFWEKAGDRGKVVQWSPQEQVLAHPATVGFLTHCGWNSTQEAISSGVPVITFPQFGDQVTNAKFLVEEFKVGVRLGRGELENRIITRDEVERALREITSGPKAEEVKENALKWKKKAEETVAKGGYSERNLVGFIEEVARKTGTK

SEQ ID NO: 2 - DNA, 1431 bp, Escherichia coli
  source 1..1431: mol_type = other DNA
atgggtagcttgacgaacaacgataatttgcacatatttttagtttgctttattgggcaaggggtggtcaatccgatgttgagacttggaaaagcattcgcctctaaagggctgttagtcaccctgtccgcacctgaaattgtaggaactgagatccggaaagcgaacaatctgaacgatgatcaacctattaaagttggtagtggtatgattcgctttgaattctttgatgacggatgggagagtgtgaacggttcgaaaccttttgatgtctgggtgtatatcaatcacctggatcagacaggtgctcagaaattaccgattatgctgaaaaagcatgaagagaccggaaccccagttagctgtcttatcctgaaccctcttgtaccctgggtcgccgatgttgcggattctttacagataccctgtgctaccttatgggttcagtcttgtgcctctttctctgcatactatcattatcatcatgggctggtacctttccctactgagtcggaacccgagattgacgtgcagttaccaggaatgccgctgcttaagtatgatgaagtaccggattacctgcatccacggactccgtacccgttttttggtactaatattttaggacaatttaaaaatctttcaaagaatttctgtattcttatggacactttctacgagctggaacatgagattatcgacaatatgtgcaaactgtgccccattaaaccgatcggcccgctgtttaaaattccgaaagacccgtccagcaacggtataactggaaactttatgaaagttgatgattgcaaggagtggttagacagtcggccgacctctacggtagtgtatgtcagtgtaggttcagtagtttatttgaaacaagagcaggtcactgagatggcgtacggcattctgaatagcgaagtaagctttttatgggtacttcgccctccatctaaacgaatcgggacagaaccacatgtgctgcctgaagaattttgggagaaggcgggagatcgtggcaaagtgctccaatggagccctcaggagcaggttcttgcacaccctgccacggttgggtttttgacacattgcggctggaactctactcaagaggcaatttctagtggtgttccggtgattacattcccacagttcggcgaccaggtaacaaacgcgaaattcctggtggaagaattcaaagtaggcgttagacttgggcgtggcgagctggaaaatcggattataacccgagacgaagttgaacgggctctgcgcgagatcacgtcaggtccaaaggcagaagaggttaaagaaaacgccttaaaatggaagaaaaaggccgaagaaaccgttgcgaaaggcggatactcagaacggaaccttgttggatttatagaggaagttgcgagaaaaacgggtaccaaa

SEQ ID NO: 3 - protein, 477 aa, Escherichia coli
MGSLTNNDNLHIFLVCFIGQGVVNPMLRLAKALASKGLLVTLSAPEIVGTEIRKANNLNDDQPIKVGSGMIRFEFFDDGWESVNGSKPFDVEVYINHLDQTGRQKLPIMLKKHEETGTPVSCLILNPLVPWVADVADSLQIPCATLWVQSCASFSAYYHYHHGLVPFPTESEPEIDVQLPGMPLLKYDEVPDYLHPRTPYPFFGTNILGQFKNLSKNFCILMDTFYELEHEIIDNMCKLCPIKPIGPLFKIPKDPSSNGITGNFMKVDDCKEWLDSRPTSTVVYVSVGSVVYLKQEQVTEMAYGILNSEVSFLWVLRPPSKRIGTEPHVLPEEFWEKAGDRGKVVQWSPQEQVLAHPATVGFLTHCGWNSTQEAISSGVPVITFPQFGDQVTNAKFLVEEFKVGVRLGRGELENRIITRDEVERALREITSGPKAEEVKENALKWKKKAEETVAKGGYSERNLVGFIEEVARKTGTK

SEQ ID NO: 4 - DNA, 27 bp, Escherichia coli
  source 1..27: mol_type = other DNA
ttagtttgctttgctgggcaaggggtg

SEQ ID NO: 5 - DNA, 27 bp, Escherichia coli
  source 1..27: mol_type = other DNA
caccccttgcccagcaaagcaaactaa

SEQ ID NO: 6 - DNA, 27 bp, Escherichia coli
  source 1..27: mol_type = other DNA
gtttgctttattgcgcaaggggtggtc

SEQ ID NO: 7 - DNA, 27 bp, Escherichia coli
  source 1..27: mol_type = other DNA
gaccaccccttgcgcaataaagcaaac

SEQ ID NO: 8 - DNA, 27 bp, Escherichia coli
  source 1..27: mol_type = other DNA
tgctttattggggcaggggtggtcaat

SEQ ID NO: 9 - DNA, 27 bp, Escherichia coli
  source 1..27: mol_type = other DNA
attgaccacccctgccccaataaagca

SEQ ID NO: 10 - DNA, 33 bp, Escherichia coli
  source 1..33: mol_type = other DNA
ttgacacattgcggcgcgaactctactcaagag

SEQ ID NO: 11 - DNA, 33 bp, Escherichia coli
  source 1..33: mol_type = other DNA
ctcttgagtagagttcgcgccgcaatgtgtcaa

SEQ ID NO: 12 - DNA, 29 bp, Escherichia coli
  source 1..29: mol_type = other DNA
cccacagttcggcgcccaggtaacaaacg

SEQ ID NO: 13 - DNA, 29 bp, Escherichia coli
  source 1..29: mol_type = other DNA
cgtttgttacctgggcgccgaactgtggg

SEQ ID NO: 14 - DNA, 27 bp, Escherichia coli
  source 1..27: mol_type = other DNA
aaaccttttgatgcctgggtgtatatc

SEQ ID NO: 15 - DNA, 27 bp, Escherichia coli
  source 1..27: mol_type = other DNA
gatatacacccaggcatcaaaaggttt

SEQ ID NO: 16 - DNA, 27 bp, Escherichia coli
  source 1..27: mol_type = other DNA
ccttttgatgtcgcggtgtatatcaat

SEQ ID NO: 17 - DNA, 27 bp, Escherichia coli
  source 1..27: mol_type = other DNA
attgatatacaccgcgacatcaaaagg

SEQ ID NO: 18 - DNA, 27 bp, Escherichia coli
  source 1..27: mol_type = other DNA
tgatgtctgggtggctatcaatcacct

SEQ ID NO: 19 - DNA, 27 bp, Escherichia coli
  source 1..27: mol_type = other DNA
aggtgattgatagccacccagacatca

SEQ ID NO: 20 - DNA, 27 bp, Escherichia coli
  source 1..27: mol_type = other DNA
ccttttgatgtcnnngtgtatatcaat

SEQ ID NO: 21 - DNA, 27 bp, Escherichia coli
  source 1..27: mol_type = other DNA
attgatatacacnnngacatcaaaagg

SEQ ID NO: 22 - DNA, 27 bp, Escherichia coli
  source 1..27: mol_type = other DNA
atgttgagacttgcaaaagcattcgcc

SEQ ID NO: 23 - DNA, 27 bp, Escherichia coli
  source 1..27: mol_type = other DNA
ggcgaatgcttttgcaagtctcaacat

SEQ ID NO: 24 - DNA, 27 bp, Escherichia coli
  source 1..27: mol_type = other DNA
cttggaaaagcattagcctctaaaggg

SEQ ID NO: 25 - DNA, 27 bp, Escherichia coli
  source 1..27: mol_type = other DNA
ccctttagaggctaatgcttttccaag

SEQ ID NO: 26 - DNA, 38 bp, Escherichia coli
  source 1..38: mol_type = other DNA
cgatgttgagacttgcaaaagcattagcctctaaaggg

SEQ ID NO: 27 - DNA, 38 bp, Escherichia coli
  source 1..38: mol_type = other DNA
ccctttagaggctaatgcttttgcaagtctcaacatcg

SEQ ID NO: 28 - DNA, 27 bp, Escherichia coli
  source 1..27: mol_type = other DNA
gtaggaactgagatgcggaaagcgaac

SEQ ID NO: 29 - DNA, 27 bp, Escherichia coli
  source 1..27: mol_type = other DNA
gttcgctttccgcatctcagttcctac

SEQ ID NO: 30 - DNA, 35 bp, Escherichia coli
  source 1..35: mol_type = other DNA
cggctggaactctactctagaggcaatttctagtg

SEQ ID NO: 31 - DNA, 35 bp, Escherichia coli
  source 1..35: mol_type = other DNA
cactagaaattgcctctagagtagagttccagccg

SEQ ID NO: 32 - DNA, 33 bp, Escherichia coli
  source 1..33: mol_type = other DNA
aaaccttttgatgtcgaggtgtatatcaatcac

SEQ ID NO: 33 - DNA, 33 bp, Escherichia coli
  source 1..33: mol_type = other DNA
gtgattgatatacacctcgacatcaaaaggttt

SEQ ID NO: 34 - DNA, 33 bp, Escherichia coli
  source 1..33: mol_type = other DNA
aaaccttttgatgtcagggtgtatatcaatcac

SEQ ID NO: 35 - DNA, 33 bp, Escherichia coli
  source 1..33: mol_type = other DNA
gtgattgatatacaccctgacatcaaaaggttt

(19) United States
(12) Patent Application Publication
(10) Pub. No.: US 2026/0242754 A1
(43) Pub. Date: Aug. 20, 2026

(54) GLYCOSYLTRANSFERASE MUTANT AND USE THEREOF

(71) Applicant: INDUSTRIAL TECHNOLOGY RESEARCH INSTITUTE, Hsinchu (TW)

(72) Inventors: Wei-Chun CHIU, Hsinchu City (TW); Hsiu-Chuan LI, Baoshan Township (TW)

(73) Assignee: INDUSTRIAL TECHNOLOGY RESEARCH INSTITUTE, Hsinchu (TW)

(21) Appl. No.: 19/436,872

(22) Filed: Dec. 30, 2025

Related U.S. Application Data
(60) Provisional application No. 63/739,727, filed on Dec. 30, 2024.

(51) Int. Cl. C12N 9/10; C12P 17/06

(52) U.S. Cl. CPC C12N 9/1051; C12P 17/06; C12Y 204/01

(57) ABSTRACT

The present disclosure provides a glycosyltransferase mutant and a use thereof. The glycosyltransferase mutant includes at least one amino acid mutation relative to the amino acid sequence of a wild-type glycosyltransferase, wherein the site of the amino acid mutation is selected from sites 30, 33, 52, 92, 372, and any combination thereof, and wherein the site of the amino acid mutation is defined based on SEQ ID NO: 1.

### CROSS REFERENCE TO RELATED APPLICATIONS

This application claims the benefit of U.S. provisional application Ser. No. 63/739,727, filed Dec. 30, 2024, the subject matter of which is incorporated herein by reference.

### REFERENCE TO AN ELECTRONIC SEQUENCE LISTING

The contents of the electronic sequence listing in ST.26 XML format (US17685-sequence listing; Size: 34,321 bytes; and Date of Creation: Dec. 26, 2025) is herein incorporated by reference in its entirety.

### TECHNICAL FIELD

The disclosure relates to a glycosyltransferase mutant and use thereof.

### BACKGROUND

Carminic acid is a currently highly important red pigment, widely used in food, cosmetics, and dyes. Current carminic acid is extracted from the bodies of cochineal insects, which entails concerns and risks such as high cost, limited sources, significant quality variations, and potential allergens.

With the prevalence of vegetarian trends and animal protection awareness, brands owners now mostly require vegan and non-animal cruelty-free products. Although Escherichia coli (E. coli) can currently be employed to produce carminic acid, the metabolic pathway for producing carminic acid in E. coli is highly complex, resulting in insufficient yields at the current stage.

Therefore, researching and developing an improved industrialized method for producing carminic acid to increase the yield of carminic acid has become an urgent need to be addressed in the field.

### SUMMARY

The present disclosure is directed to mutants of a wild-type glycosyltransferase of Escherichia coli subjected to site-specific mutations, particularly mutations directed to amino acids associated with the substrate-binding pocket or the substrate (i.e., kermesic acid) entry and release channels, in order to improve the catalytic activity of the glycosyltransferase and thereby increase the yield of carminic acid.

According to some embodiments, the present disclosure provides a glycosyltransferase mutant. The glycosyltransferase mutant comprises at least one amino acid mutation relative to an amino acid sequence of the wild-type glycosyltransferase, wherein a site of the amino acid mutation is selected from a group consisting of sites 30, 33, 52, 92, 372, and any combination thereof, and wherein the site of the amino acid mutation is defined based on an amino acid sequence of SEQ ID NO: 1.

According to some embodiments, the present disclosure provides a method for catalyzing a glycosylation reaction, comprising utilizing the aforementioned glycosyltransferase mutant. The glycosylation reaction is, in particular, a glycosylation reaction in Escherichia coli.

In order to provide a better understanding of the above and other aspects of the present disclosure, embodiments are given below and described in detail in conjunction with the accompanying drawings as follows.

### BRIEF DESCRIPTION OF THE DRAWINGS

FIG. 1 illustrates a predicted three-dimensional protein structure of a wild-type glycosyltransferase.

FIGS. 2A-2B illustrate schematic diagrams of docking simulations between the wild-type glycosyltransferase and kermesic acid as the substrate and uridine diphosphate glucose (UDP-glucose) as the glycosyl donor.

FIG. 3 illustrates experimental results of alanine scanning performed on amino acids surrounding the substrate-binding region.

FIG. 4 illustrates a schematic diagram showing the positions of some potential candidate residues on the wild-type glycosyltransferase.

FIG. 5A illustrates a schematic diagram of a channel through which the substrate, kermesic acid, enters and exits the catalytic center.

FIG. 5B illustrates the enzyme activity test results of mutations performed on amino acids located at the periphery of the substrate-binding region.

FIG. 6A illustrates the protein expression results of the wild-type glycosyltransferase and a glycosyltransferase mutant with three-site mutations.

FIG. 6B illustrates the enzyme activity evaluation results of the wild-type glycosyltransferase and the glycosyltransferase mutant with three-site mutations.

FIG. 6C illustrates the enzyme kinetic parameter determination results of the wild-type glycosyltransferase and the glycosyltransferase mutant with three-site mutations.

FIG. 7 illustrates the results of protein structural stability analysis based on Differential Scanning calorimetry (DSC).

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

Various embodiments will be described in greater detail below in conjunction with the accompanying drawings. It should be understood that the following description provides many different embodiments or examples to implement different aspects of the present disclosure, and the descriptions and drawings are provided for illustration purposes only and are not intended to be limiting. For the sake of clarity, some elements and/or symbols may be omitted in some drawings. Furthermore, the elements in the drawings are primarily used to clearly display the features intended to be presented in the present disclosure and may not be drawn to actual scale. It is contemplated that elements and features in one embodiment, if they can be advantageously incorporated into another embodiment, will not be redundantly described in the other embodiment. On the other hand, in the present disclosure, the term “about” means that a specified amount may increase or decrease by an amount that a person skilled in the art would recognize as a common and reasonable magnitude.

In the metabolic production pathway for producing carminic acid in Escherichia coli, the final step is the catalysis of the intermediate product kermesic acid by a uridine diphosphate (UDP)-dependent glycosyltransferase GtGCT. The glycosyltransferase GtGCT uses uridine diphosphate glucose (UDP-glucose) as a glycosyl donor to specifically transfer a sugar molecule to the glycosylation site of an acceptor molecule (i.e., kermesic acid) to form the final product, carminic acid, as shown in the following Formula 1.

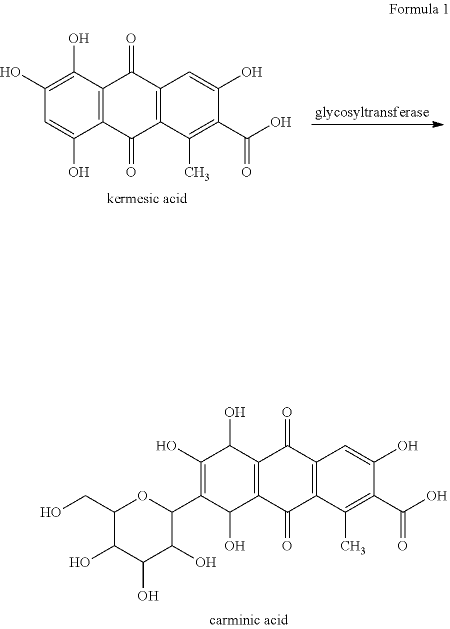 [SMILES: Cc1c(C(=O)O)c(O)cc2c1C(=O)c1c(O)cc(O)c(O)c1C2=O ; Cc1c(C(=O)O)c(O)cc2c1C(=O)C1=C(C2=O)C(O)C(O)=C(C2OC(CO)C(O)C(O)C2O)C1O]

The present disclosure relates to enzyme design based on protein structure to optimize a key reaction enzyme (i.e., glycosyltransferase) in the metabolic pathway of Escherichia coli for producing carminic acid, thereby forming glycosyltransferase mutants with the goal of increasing the yield of carminic acid.

According to some embodiments, the present disclosure provides a glycosyltransferase mutant. The glycosyltransferase mutant comprises at least one amino acid mutation relative to the amino acid sequence of a wild-type glycosyltransferase, wherein the site of the amino acid mutation is selected from the group consisting of sites 30, 33, 52, 92, 372 and any combination thereof, and wherein the site of the amino acid mutation is defined based on the amino acid sequence of SEQ ID NO: 1. That is, the site of the amino acid mutation of the glycosyltransferase mutant may be a single site or multiple sites, such as a single mutation at site 30, a single mutation at site 33, a single mutation at site 92, a double-site mutation at sites 30 and 33, a double-site mutation at sites 33 and 92, a three-site mutation at sites 30, 33, and 92, a four-site mutation at sites 30, 33, 52, and 92, or mutations at other suitable sites.

According to some embodiments, the aforementioned amino acid mutation site is selected from the group consisting of: (1) substitution of G at site 30, (2) substitution of F at site 33, (3) substitution of I at site 52, (4) substitution of W at site 92, (5) substitution of Q at site 372, and (6) any combination of the substitution modes of (1) to (5). That is, the amino acid substitution site of the glycosyltransferase mutant may be a single site or multiple sites, such as a single substitution at site 30, a single substitution at site 33, a single substitution at site 92, a double-site substitution at sites 30 and 33, a double-site substitution at sites 33 and 92, a three-site substitution at sites 30, 33, and 92, a four-site substitution at sites 30, 33, 52, and 92, or substitutions at other suitable sites.

According to some embodiments, G at site 30 of the wild-type glycosyltransferase is substituted by A (i.e., G30A), to form the glycosyltransferase mutant of the present disclosure.

According to some embodiments, F at site 33 of the wild-type glycosyltransferase is substituted by L (i.e., F33L), to form the glycosyltransferase mutant of the present disclosure.

According to some embodiments, I at site 52 of the wild-type glycosyltransferase is substituted by M (i.e., I52M), to form the glycosyltransferase mutant of the present disclosure.

According to some embodiments, W at site 92 of the wild-type glycosyltransferase is substituted by one of the following amino acids: V, P, L, N, R, H, G, S, Y, D, K, A, E, Q, M, F, C and I (i.e., W92V, W92P, W92L, W92N, W92R, W92H, W92G, W92S, W92Y, W92D, W92K, W92A, W92E, W92Q, W92M, W92F, W92C and W921), to form the glycosyltransferase mutant of the present disclosure.

According to some embodiments, Q at site 372 of the wild-type glycosyltransferase is substituted by L (i.e., Q372L), to form the glycosyltransferase mutant of the present disclosure.

According to some embodiments, when W at site 92 of the wild-type glycosyltransferase is substituted by E, the enzymatic catalytic activity of the formed glycosyltransferase mutant (i.e., W92E) is at least 4 times that of the wild-type glycosyltransferase, for example, 4.6 times.

According to some embodiments, when G at site 30 of the wild-type glycosyltransferase is substituted by A, and F at site 33 is substituted by L, the enzymatic catalytic activity of the formed glycosyltransferase mutant is at least 3 times that of the wild-type glycosyltransferase, for example, 3.7 times.

According to some embodiments, when G at site 30 of the wild-type glycosyltransferase is substituted by A, F at site 33 is substituted by L, and W at site 92 is substituted by E, the enzymatic catalytic activity of the formed glycosyltransferase mutant is at least 12 times that of the wild-type glycosyltransferase, for example, 12.3 times.

According to some embodiments, when G at site 30 of the wild-type glycosyltransferase is substituted by A, F at site 33 is substituted by L, and W at site 92 is substituted by E, the formed glycosyltransferase mutant has a melting temperature (Tm) at least 2° C. higher than that of the wild-type glycosyltransferase, wherein the melting temperature of the wild-type glycosyltransferase is, for example, 53.07° C.

According to some embodiments, the present disclosure provides a use of a glycosyltransferase mutant for catalyzing a glycosylation reaction, particularly a glycosylation reaction in Escherichia coli.

According to some embodiments, the glycosylation reaction is used for the production of carminic acid.

To make the aforementioned and other objects, features, and advantages of the present disclosure more clearly understood, several embodiments are specifically listed below for detailed description:

### EXAMPLES

### Example 1: Protein Structure Prediction of Glycosyltransferase

The wild-type glycosyltransferase used in the present disclosure is Gentiana triflora glycosyltransferase (GtGCT), which has the amino acid sequence defined by SEQ ID NO: 1 (Uniprot ID: A0A0B6VIJ5). The amino acid sequence defined by SEQ ID NO: 1 has 477 amino acids and a predicted molecular weight of 60.7 kDa.

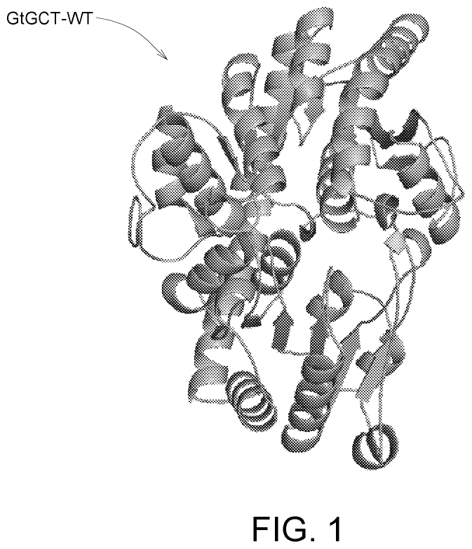

The amino acid sequence defined by SEQ ID NO: 1 was input into the Google Colab platform, and after performing computations with the AlphaFold2 program (URL: https://colab.research.google.com/github/sokrypton/ColabFold/blob/main/Alph aFold2.ipynb), the predicted three-dimensional protein structure of the wild-type glycosyltransferase GtGCT-WT, as shown in FIG. 1, was obtained.

### Example 2: Molecular Docking of the Protein Structure of Wild-Type Glycosyltransferase GtGCT-WT with Substrate (i.e., Kermesic Acid) and Uridine Diphosphate Glucose (UDP-Glucose)

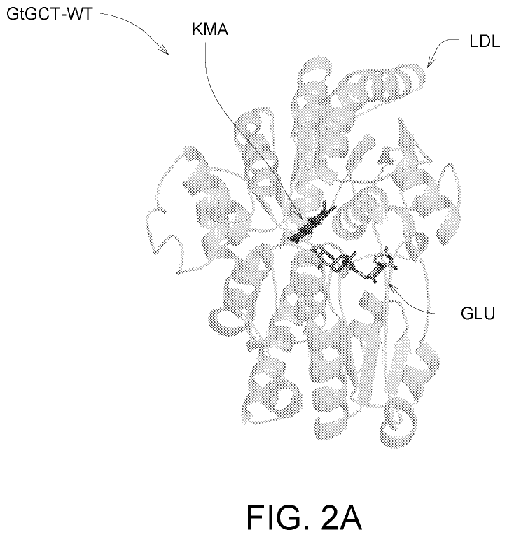

After predicting the protein structure of the wild-type glycosyltransferase GtGCT-WT using the aforementioned AlphaFold2, a C-glycosyltransferase was used as a template (PDB database, No. 6JTD) to identify potential substrate binding pocket regions in the wild-type glycosyltransferase GtGCT-WT. A cubic box of appropriate size was set, and then the substrate (i.e., kermesic acid) and uridine diphosphate glucose were input into AUTODOCK software for docking computations. The optimal spatial orientation and pose obtained after computation were also the results with the lowest free energy, as shown in FIG. 2A and FIG. 2B. FIG. 2A and FIG. 2B illustrate schematic diagrams of docking simulations between the wild-type glycosyltransferase GtGCT-WT and kermesic acid KMA as a substrate and uridine diphosphate glucose GLU as a glycosyl donor.

### Example 3: Alanine Screening of Amino Acids in the Vicinity of the Substrate-Binding Pocket

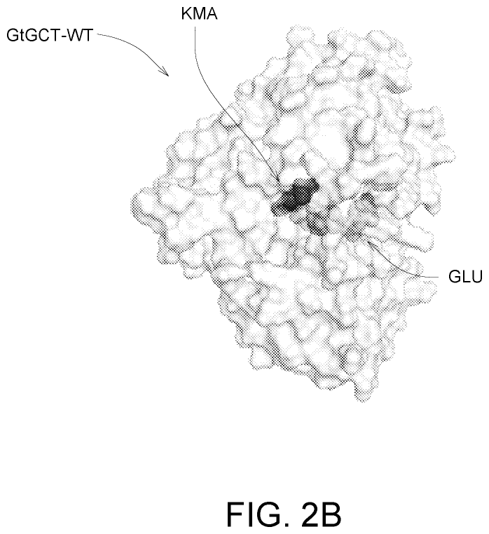

Please refer again to FIG. 2A and FIG. 2B. According to the prediction results, the structure of the wild-type glycosyltransferase GtGCT-WT is an α/β Rossmann-type fold and possesses a flexible lid loop LDL. The lid loop LDL is the structure responsible for the opening and closing of the entry and exit of the substrate (i.e., kermesic acid); therefore, it can be inferred that amino acids associated with the lid loop LDL exert a certain degree of influence on the substrate (i.e., kermesic acid). Amino acids associated with the lid loop LDL are, for example, V91, W92 and Y94. Additionally, other amino acids associated with the substrate-binding pocket (for example, I18, G19, Q20, W368 and D389) may also play a key role. Further experiments involving alanine screening of the aforementioned amino acid sites of interest were conducted, and the results are shown in FIG. 3.

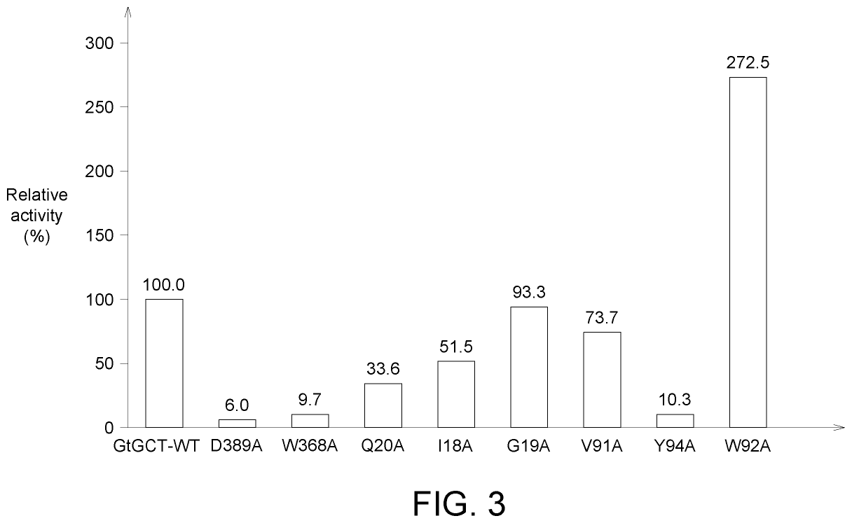

Please refer to FIG. 3, which illustrates the experimental results of alanine screening performed on amino acids in the vicinity of the substrate-binding region. Please also refer in conjunction to FIG. 4, which illustrates the positions of some amino acids of interest on the wild-type glycosyltransferase GtGCT-WT.

In brief, alanine screening involves first replacing the aforementioned amino acids of interest I18, G19, Q20, W368, D389, V91, W92 and Y94 respectively with alanine (A), subsequently expressing and purifying these glycosyltransferase mutants I18A, G19A, Q20A, W368A, D389A, V91A, W92A and Y94A using an Escherichia coli system, and comparing the enzymatic activity with the wild-type glycosyltransferase GtGCT. The glycosyltransferase mutants I18A, G19A, Q20A, W368A, D389A, V91A, W92A and Y94A are prepared and purified in a manner similar to the preparation and purification of the glycosyltransferase mutant GtGCT-G30A/F33L/W92E as described below, with the only difference being the primer sequences for the mutation sites; other identical parts will not be described in detail.

In the present embodiment, the sequences of the forward primers and reverse primers of the glycosyltransferase mutants I18A, G19A, Q20A, W368A, D389A, V91A, W92A and Y94A are shown in the following table as SEQ ID NO: 4 to SEQ ID NO: 19, respectively.

TABLE 1

| Mutation Site | Primer Name | SEQ ID NO |
|---|---|---|
| I18A | I18A-F′ primer | 4 |
| | I18A-R′ primer | 5 |
| G19A | G19A-F′ primer | 6 |
| | G19A-R′ primer | 7 |
| Q20A | Q20A-F′ primer | 8 |
| | Q20A-R′ primer | 9 |
| W368A | W368A-F′ primer | 10 |
| | W368A-R′ primer | 11 |
| D389A | D389A-F′ primer | 12 |
| | D389A-R′ primer | 13 |
| V91A | V91A-F′ primer | 14 |
| | V91A-R′ primer | 15 |
| W92A | W92A-F′ primer | 16 |
| | W92A-R′ primer | 17 |
| Y94A | Y94A-F′ primer | 18 |
| | Y94A-R′ primer | 19 |

Determination of enzymatic activity was completed using the UDP-Glo Glycosyltransferase Assay kit (Promega) according to the conditions described in the user manual. The principle is briefly described as follows: after the glycosyltransferase reacts with the substrate, a UDP detection reagent is added, which converts UDP into ATP, and the newly synthesized ATP is measured using a luciferase reaction. The generated luminescence signal is positively correlated with the amount of UDP produced by the glycosyltransferase; therefore, the luminescence signal represents the activity of the glycosyltransferase. The test conditions are as follows: enzyme concentration of 5 μg, uridine diphosphate glucose concentration of 0.5 mM, kermesic acid concentration of 0.5 mM, Tris-HCl (pH 8.0) concentration of 100 mM, reaction volume of 25 μl, reaction temperature of 30° C., and reaction time of 60 minutes; subsequently, UDP detection reagent (concentration of 25 μl) was added, reaction temperature was 25° C., and reaction time was 60 minutes; then, the luminescence signal was directly detected using the Promega GloMax Discover System.

As shown in FIG. 3, when site W92 is mutated to A (i.e., W92A), its ability to enzymatically catalyze the substrate can be increased by approximately 2.7 times relative to the wild-type glycosyltransferase, indicating that mutations at this site, particularly mutation to A, can indeed affect the catalytic ability of the enzyme.

### Example 4: Site-Saturation Mutagenesis of W92

Following the verification in Example 3 that the mutation of site W92 to A can affect the enzymatic catalytic ability of the glycosyltransferase, a site-saturation mutagenesis test was further performed on site W92. Please refer to Table 2 below, which shows the experimental results after performing site-saturation mutagenesis on position W92 of the wild-type glycosyltransferase GtGCT-WT.

Site-saturation mutagenesis can introduce all possible mutations by systematically changing the amino acid at a specific site while simultaneously analyzing the impact of each variation on protein function. The method for preparing the site-saturation variant library is as follows: designing saturation mutation primers for position W92 (the sequences of the forward primer and reverse primer are SEQ ID NO: 20 and SEQ ID NO: 21, respectively, where “N” in the sequences represents any of the nucleotides A, C, G, or T); using the Agilent QuikChange Site-Directed Mutagenesis Kit; and performing the collection of mutant DNA according to the operation method described in the manual. Subsequently, the collected DNA library was transformed into competent cells (purchased from UNI-ONWARD Corp., brand: YB Biotech, model: ECOS-21 Economic type [BL-21(DE3)]); colonies were randomly selected, and after confirming the mutation results by DNA sequencing, the mutant enzyme proteins were expressed, and the enzymatic activities of the formed glycosyltransferase mutants W92V, W92P, W92L, W92N, W92R, W92H, W92G, W92S, W92Y, W92D, W92K, W92A, W92E, W92Q, W92M, W92F, W92T, W92C and W921 were compared with the wild-type glycosyltransferase GtGCT-WT (the measurement method for enzymatic activity is as previously described).

In Table 2, “+” indicates that the enzymatic activity of the glycosyltransferase mutant is between 1 to 2 times that of the wild-type glycosyltransferase GtGCT-WT; “++” indicates that the enzymatic activity of the glycosyltransferase mutant is greater than 2 times and equal to or less than 3 times that of the wild-type glycosyltransferase GtGCT-WT. “+++” indicates that the enzymatic activity of the glycosyltransferase mutant is greater than 3 times and equal to or less than 4 times that of the wild-type glycosyltransferase GtGCT-WT. “++++” indicates that the enzymatic activity of the glycosyltransferase mutant is greater than 4 times that of the wild-type glycosyltransferase GtGCT-WT. “−” indicates that the enzymatic activity of the glycosyltransferase mutant is not higher than that of the wild-type glycosyltransferase GtGCT-WT.

TABLE 2

| | Enzymatic activity relative |
|---|---|
| Glycosyltransferase | to wild-type |
| Mutant | glycosyltransferase |
| W92V | + |
| W92P | ++ |
| W92L | + |
| W92N | ++ |
| W92R | +++ |
| W92H | + |
| W92G | ++ |
| W92S | + |
| W92Y | + |
| W92D | ++ |
| W92K | ++ |
| W92A | +++ |
| W92E | ++++ |
| W92Q | ++ |
| W92M | + |
| W92F | + |
| W92T | − |
| W92C | + |
| W92I | + |

Please refer to Table 1. Among the mutation positions of W92, all mutations except for the mutation to T can increase enzymatic activity; W92R and W92A can reach more than 3 times that of the wild-type glycosyltransferase GtGCT-WT. The optimal mutation is W92E, which has an enzymatic activity relative to the wild-type glycosyltransferase GtGCT-WT of at least 4 times, or even more than 4.6 times.

Clearly, among the W92E mutation position, mutations to E, R, and A most effectively increase enzymatic activity. A possible reason is that tryptophan (W) is the largest of all amino acids and is relatively rigid in nature. When it is mutated to E, R, and A, the volume can be reduced to decrease steric hindrance in the structure of the lid loop, while making the properties of the lid loop softer and more flexible, allowing the substrate to enter the enzyme more easily for reaction.

### Example 5: Study of Substrate Entry and Release Channels of Glycosyltransferase

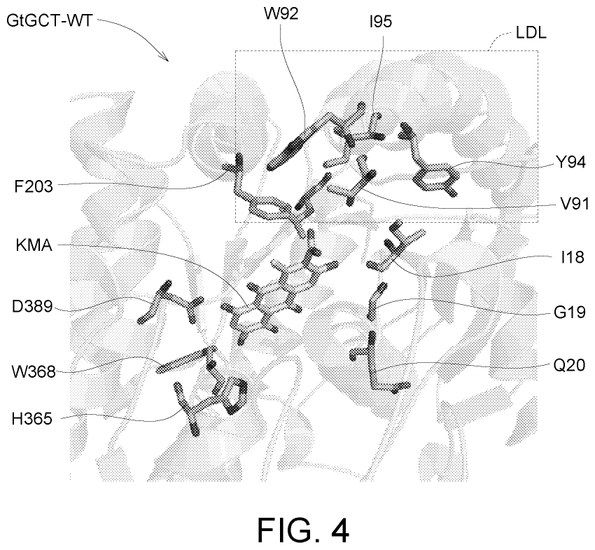

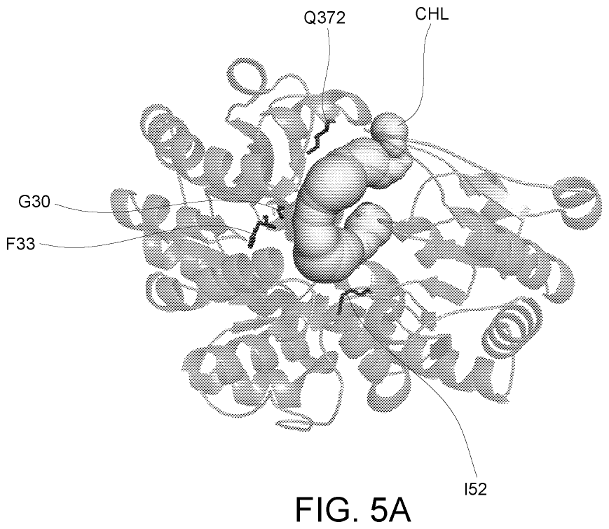

In enzyme design for increasing enzymatic activity, the channels for substrate entry and release are very important. These channels not only affect the binding efficiency of the substrate but also the catalytic efficiency of the enzyme and product release. The structure and size of the substrate entry channel directly affect how efficiently the substrate binds to the active site of the enzyme. If the channel is too small or improperly shaped, it may restrict substrate entry, thereby reducing the catalytic efficiency of the enzyme. For example, in certain enzymes, the steric hindrance of specific amino acid residues can affect substrate binding, leading to a decrease in enzymatic activity. Please refer to FIG. 5A. In the present embodiment, the channel CHL for the entry of kermesic acid KMA as a substrate into the substrate catalytic center for the catalytic reaction and its subsequent release is calculated and depicted using software, thereby evaluating amino acids surrounding this channel CHL that may affect it, such as the four amino acids G30, F33, 152 and Q372. Subsequently, mutations are performed on these four amino acids, and their enzymatic activities are tested (as shown in FIG. 5B).

Please refer to FIG. 5B, which illustrates the enzyme activity test results for mutations performed on amino acids at the periphery of the substrate-binding region (for example, the channel for substrate entry and release within the enzyme).

As shown in FIG. 5B, sites G30 and F33 are respectively mutated to A and L to form glycosyltransferase mutants G30A and F33L. In G30A, A is a hydrophilic amino acid while A is a hydrophobic amino acid; the mutation of G to A can allow the substrate and enzyme to form better hydrophobic interactions. In F33L, although both F (Phenylalanine) and L (Leucine) are hydrophobic amino acids, L has a smaller volume, which reduces steric hindrance and should allow the substrate to enter and release more easily. On the other hand, sites I52 and Q372 are respectively mutated to M and L to form glycosyltransferase mutants I52M and Q372L. The glycosyltransferase mutants G30A, F33L, I52M and Q372L are prepared and purified in a manner similar to the glycosyltransferase mutant G30A/F33L/W92E described below, with the only difference being the primer sequences used to prepare the mutation sites; other identical parts will not be described in detail. The enzymatic activity test method is as previously described. In the present embodiment, the sequences of the forward and reverse primers for the glycosyltransferase mutants G30A, F33L, G30A/F33L, I52M and Q372L are shown in the following table as SEQ ID NO: 22 to SEQ ID NO: 31, respectively.

TABLE 3

| Mutation Site | Primer Name | SEQ ID NO |
|---|---|---|
| G30A | G30A-F′ primer | 22 |
| | G30A-R′ primer | 23 |
| F33L | F33L-F′ primer | 24 |
| | F33L-R′ primer | 25 |
| G30A/F33L | G30A/F33L-F′ primer | 26 |
| | G30A/F33L-R′ primer | 27 |
| I52M | I52M-F′ primer | 28 |
| | I52M-R′ primer | 29 |
| Q372L | Q372L-F′ primer | 30 |
| | Q372L-R′primer | 31 |

From the results in FIG. 5B, it can be seen that the single mutants G30A, F33L, I52M and Q372L of the glycosyltransferase all exhibit increased enzymatic activity relative to the wild-type glycosyltransferase GtGCT-WT, for example, increasing enzymatic activity by approximately 1.38, 1.4, 1.29, and 1.7 times, respectively. Furthermore, the double mutant G30A/F33L of the glycosyltransferase, formed by combining two mutation sites, can increase enzymatic activity by 3.7 times. It is evident that the four amino acids G30, F33, 152 and Q372 surrounding the screened channel can enhance enzymatic activity relative to the wild-type glycosyltransferase GtGCT-WT, whether as single mutations or double mutations.

### Example 6: Calculation of Free Energy for Molecular Docking of Wild-Type Glycosyltransferase GtGCT-WT and Various Glycosyltransferase Mutants

Using the optimal spatial orientation and position obtained from the aforementioned AUTODOCK software as a template, optimization calculations of structures and mutated amino acids were subsequently performed using Accelrys Discovery Studio 2.1 software. Based on a comparison of the resulting minimum free energy changes with the unaltered wild-type glycosyltransferase GtGCT-WT, potential candidate mutation positions were selected as shown in Table 4. In Table 4, a lower (more negative) binding energy value represents more stable binding and higher affinity.

TABLE 4

| | Binding Energy with Kermesic Acid |
|---|---|
| Enzyme | Substrate (kcal/mol) |
| GtGCT-WT | −6.15 |
| GtGCT-H365A | −5.41 |
| GtGCT-D389A | −4.94 |
| GtGCT-I18A | −6.02 |
| GtGCT-I19A | −6.04 |
| GtGCT-V22A | −5.51 |
| GtGCT-L128A | −5.9 |
| GtGCT-F203A | −5.89 |
| GtGCT-I95M | −6.27 |
| GtGCT-I52M | −6.23 |
| GtGCT-I95A | −6.24 |
| GtGCT-W92E | −6.23 |
| GtGCT-G30A | −6.19 |
| GtGCT-F33L | −6.14 |
| GtGCT-T359I | −6.05 |
| GtGCT-V360A | −6.01 |
| GtGCT-Q372L | −6.14 |

Summarizing the above experimental results, the site-saturation mutagenesis test of W92 determined that a change in W92E can increase enzymatic activity by approximately 4.5 times. Through channel analysis of substrate entry and release, it was determined that single-site mutations of G30A and F33L can increase enzymatic activity by approximately 1.38 times and approximately 1.4 times, respectively. Further combining the two mutation sites G30A and F33L to form a double-site glycosyltransferase mutant can increase enzymatic activity by up to 3.7 times.

### Example 7: Preparation of Triple-Site Glycosyltransferase Mutants and Investigation of Enzyme Protein Purification, Activity, and Enzyme Kinetic Parameters

### Example 7-1: Construction of Expression Vectors for Wild-Type Glycosyltransferase GtGCT-WT and Triple-Site Glycosyltransferase Mutant G30A/F33L/W92E

The amino acid sequence (Uniprot ID: A0A0B6VIJ5) of the wild-type glycosyltransferase GtGCT-WT of the aforementioned SEQ ID NO: 1 was translated into a cDNA sequence to form SEQ ID NO: 2, and restriction enzyme sites for AscI and NotI were added to the front and back ends of this sequence. cDNA synthesis was commissioned to Twist Bioscience. Subsequently, this segment of cDNA was fused with the protein expression vector pET28a using the AscI and NotI restriction enzyme sites to become the wild-type DNA construct pET28a-GtGCT-WT. SEQ ID NO: 3 is the amino acid sequence of the triple-mutant glycosyltransferase mutant GtGCT-G30A/F33L/W92E. The mutation sites G30A, F33L, and W92E were generated using SEQ ID NO: 22 and SEQ ID NO: 23, SEQ ID NO: 24 and SEQ ID NO: 25, and SEQ ID NO: 32 and SEQ ID NO: 33, respectively, as mutation primers. Point mutations were performed referring to the manual method of the Agilent QuikChange Site-Directed Mutagenesis Kit to become the mutant DNA construct pET28a-GtGCT-G30A/F33L/W92E (SEQ ID NO: 3).

### Example 7-2: Expression and Purification of Enzyme Protein

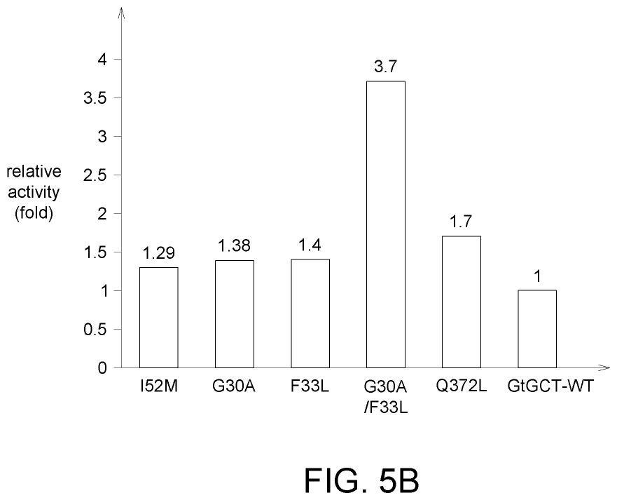

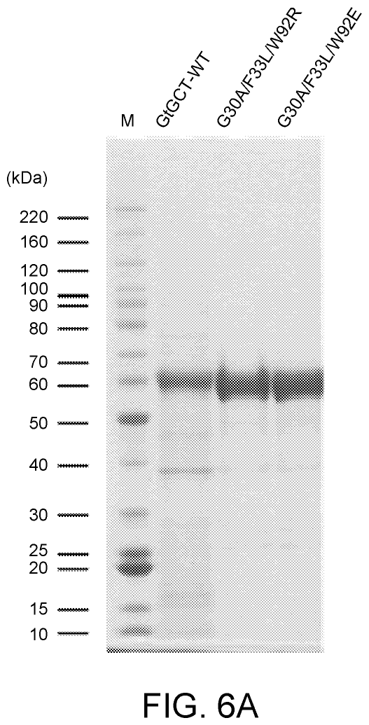

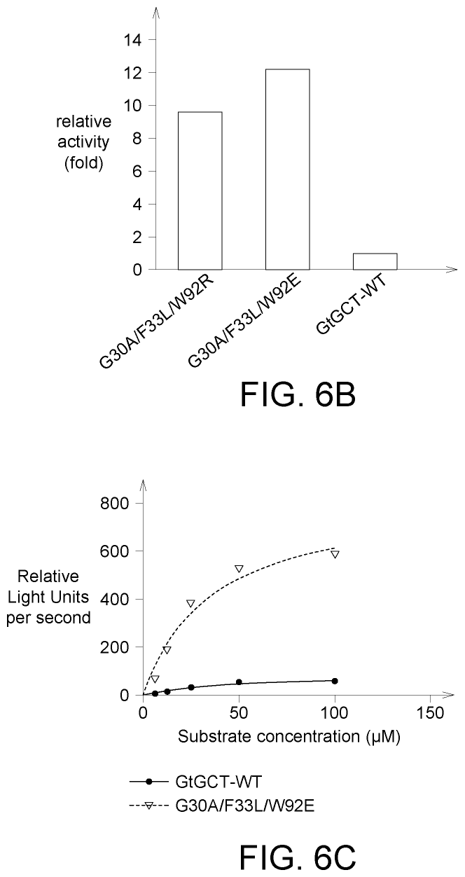

The aforementioned wild-type DNA construct pET28a-GtGCT-WT or mutant DNA construct pET28a-GtGCT-G30A/F33L/W92E was introduced into the protein expression competent cell BL21(DE3) (purchased from UNI-ONWARD Corp., brand: YB Biotech, model: ECOS-21 Economic type [BL-21(DE3)]) via heat shock. A single colony was selected from a culture plate containing the selection antibiotic Kanamycin and cultured in 5 ml of LB medium (14 hours, 37° C.). The following day, 500 μl of the bacterial culture was taken for scaled-up cultivation in 50 ml of fresh LB medium (2 hours, 37° C.). 1 mM IPTG inducer was added, and cultivation continued at 20° C. for 14 hours. Thereafter, the bacterial culture was collected by centrifugation, and the cells were lysed using a cell lysis reagent (Sigma-Aldrich CelLytic B Cell Lysis Reagent). Subsequently, the supernatant was collected after high-speed centrifugation and subjected to affinity binding purification through a protein purification column (GE Healthcare Ni-Sepharose excel column). The purification steps were performed according to the column user manual. Finally, the enzyme protein was eluted using an elution buffer containing 150 mM Tris-HCl (pH 8.0), 200 mM sodium chloride (NaCl), and 250 mM imidazole. The enzyme protein was then analyzed by SDS-PAGE, as shown in FIG. 6A.

Please refer to FIG. 6A, which illustrates the enzyme protein expression results of the wild-type glycosyltransferase GtGCT-WT and the triple-site glycosyltransferase mutants G30A/F33L/W92E and G30A/F33L/W92R. The glycosyltransferase mutant G30A/F33L/W92R was prepared and purified in a manner similar to the aforementioned method for glycosyltransferase mutant G30A/F33L/W92E, with the only difference being the primer sequences for the mutation sites of W92E and W92R. Other identical parts will not be described in detail. The forward and reverse primers for the mutation site of W92R are SEQ ID NO: 34 and SEQ ID NO: 35, respectively.

As shown in FIG. 6A, under SDS-PAGE analysis, a major band was obtained at the 60 kDa position relative to the molecular weight marker M, which represents the enzyme protein of the wild-type and mutant strains. In the SDS-PAGE analysis results, the enzyme protein bands of glycosyltransferase mutants GtGCT-G30A/F33L/W92E and GtGCT-G30A/F33L/W92R are both thicker than the enzyme protein band of the wild-type glycosyltransferase GtGCT-WT, indicating that the enzyme proteins of the triple-site glycosyltransferase mutants GtGCT-G30A/F33L/W92E and GtGCT-G30A/F33L/W92R have higher expression levels.

### Example 7-3: Enzyme Protein Activity

FIG. 6B illustrates the enzyme protein activity evaluation results of the wild-type glycosyltransferase GtGCT-WT and the triple-site glycosyltransferase mutants G30A/F33L/W92E and G30A/F33L/W92R. The method for determining enzymatic activity is as previously described.

As shown in FIG. 6B, the triple-site glycosyltransferase mutant GtGCT-G30A/F33L/W92E can increase enzymatic activity by up to 12 times relative to the wild-type glycosyltransferase GtGCT-WT, while the triple-site glycosyltransferase mutant G30A/F33L/W92R can also increase enzymatic activity by approximately 9.5 times.

### Example 7-4: Determination of Enzyme Kinetic Parameters

By determining kinetic parameters, the mechanism of action of an enzyme can be deduced. For example, using the Michaelis-Menten equation, the process of enzyme-substrate binding and its rate of conversion into product can be analyzed. This information is of great significance for designing enzymes or improving enzymatic catalytic processes. The steady-state kinetic analysis of the wild-type and mutant strains of the present disclosure was performed using the UDP-Glo Glycosyltransferase Assay Kit from Promega according to its manual steps.

In brief, the enzyme (at a reaction concentration of 0.1 μM) of the wild-type glycosyltransferase GtGCT-WT and the triple-site glycosyltransferase mutant GtGCT-G30A/F33L/W92E was added to different concentrations of substrate (6.25, 12.5, 25, 50, 100 mM), uridine diphosphate glucose (0.5 mM), and Tris-HCl (pH 8.0, 100 mM), with a reaction volume of 25 μl, a reaction temperature of 30° C., and a reaction time of 30 minutes. Thereafter, 25 μl of UDP test reagent was added at a reaction temperature of 25° C. for a reaction time of 60 minutes, followed by direct detection of the luminescence signal using the Promega GloMax Discover System. The Vmax and Km parameters were calculated using non-linear regression of the Michaelis-Menten equation in GraphPad Prism 6, and the results are shown in FIG. 6C and Table 5. Vmax represents the maximum reaction rate, Km represents the Michaelis constant (affinity between enzyme and substrate), kcat represents the catalytic constant (enzyme catalytic efficiency), and kcat/Km is used to measure the enzymatic catalytic efficiency.

TABLE 5

| | Vmax | | | |
|---|---|---|---|---|
| | [Relative | | | |
| | Light | | | |
| | Units per | | | |
| | second: | Km | Kcat | Kcat/Km |
| Enzyme | RLU/sec] | [μM] | [s⁻¹] | [s⁻¹μM⁻¹] |
| wild-type | 93.1 ± 12.6 | 51.2 ± 14.3 | 31.6 ± 1.7 | 0.6 |
| glycosyl- | | | | |
| transferase | | | | |
| GtGCT-WT | | | | |
| glycosyl- | 837.2 ± 66.4 | 36.5 ± 6.7 | 283.8 ± 6.7 | 7.8 |
| transferase | | | | |
| mutant G30A/ | | | | |
| F33L/W92E | | | | |

From the results of FIG. 6C and Table 5, it can be seen that the Km of the wild-type glycosyltransferase GtGCT-WT is 51.2 mM, while the Km value of the glycosyltransferase mutant G30A/F33L/W92E is 36.5 mM, representing that the substrate of the mutant strain has a better affinity for the enzyme within the binding pocket compared to the wild-type strain. In terms of catalytic rate, the kcat of the wild-type glycosyltransferase GtGCT-WT is 31.6/sec, while the kcat value of the glycosyltransferase mutant G30A/F33L/W92E is 283.8/sec, representing that the enzymatic catalytic efficiency of the glycosyltransferase mutant G30A/F33L/W92E is higher than that of the wild-type glycosyltransferase GtGCT-WT. Overall, the enzymatic catalytic efficiency (kcat/Km) of the glycosyltransferase mutant G30A/F33L/W92E is 7.8 s⁻¹ μM⁻¹, which is an increase of at least 13 times compared to the kcat/Km of 0.6 s⁻¹ μM⁻¹ for the wild-type glycosyltransferase GtGCT-WT.

### Example 8: Investigation of Protein Structural Stability

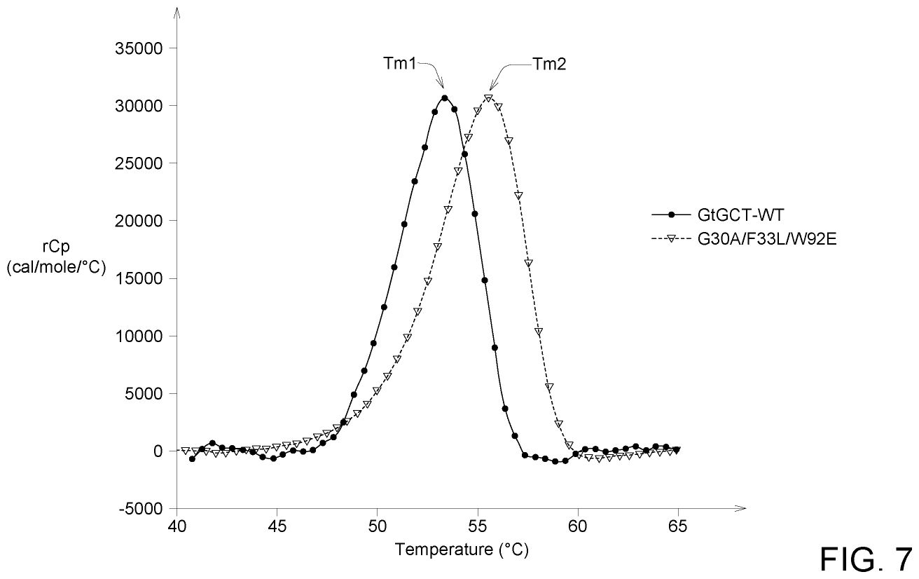

FIG. 7 illustrates the results of protein structural stability analysis based on Differential Scanning calorimetry (DSC).

Differential Scanning calorimetry is an instrument used to measure energy changes in a protein sample under specific temperature conditions, including the release and absorption of heat. When a protein is in its natural state in solution, it is in a folded state; however, upon denaturation, it forms an unfolded state, at which time heat is released and detected by DSC. A higher temperature required to cause the unfolded state indicates a more stable structure of the protein. Based on this principle, the structural stabilities of the wild-type glycosyltransferase GtGCT-WT and the glycosyltransferase mutant G30A/F33L/W92E were tested and compared. Prior to testing protein samples, a buffer baseline must first be established. Bubbles are removed from the same buffer used for protein dilution using a vacuum pump, and then the buffer is injected into the DSC sample chamber using a sample syringe. Scanning conditions are set with a starting temperature of 20° C., an ending temperature of 100° C., and a temperature ramp rate of 60° C./hour until all buffer baselines reach equilibrium. Thereafter, bubbles are removed from a protein sample with a protein concentration of approximately 1 mg/ml using a vacuum pump, and the sample is injected into the DSC sample chamber using a sample syringe. The setting conditions are the same as those for the buffer baseline scan. The melting temperature (Tm) of the sample is obtained from the resulting data using the GE Healthcare MicroCal VP-DSC analysis program, as shown in FIG. 7. The Y-axis represents the excess heat capacity rCp (cal/mole/° C.).

According to the profile shown in the DSC analysis of FIG. 7, it can be seen that during the heating and unfolding process of the GtGCT enzyme protein, only one major melting peak is generated. This represents that the structure of this protein participates in the unfolding process under “one single” identical domain. The interacting forces within this domain are very strong and do not separate during the heat treatment process. This result is completely consistent with the prediction by AlphaFold2 that the GtGCT enzyme protein is an α/β Rossmann-type fold.

Furthermore, it can also be seen from FIG. 7 that in terms of melting temperature, the melting temperature Tm1 of the wild-type glycosyltransferase GtGCT-WT is approximately 53.07° C., while the melting temperature Tm2 of the glycosyltransferase mutant G30A/F33L/W92E is approximately 55.13° C. The melting temperature Tm2 of the glycosyltransferase mutant G30A/F33L/W92E is 2.06° C. higher than that of the wild-type glycosyltransferase GtGCT-WT, proving that the protein structural stability of the glycosyltransferase mutant G30A/F33L/W92E is indeed higher than that of the wild-type glycosyltransferase GtGCT-WT.

In summary, relative to the wild-type glycosyltransferase, the glycosyltransferase mutant according to some embodiments of the present disclosure can possess higher enzymatic catalytic activity and better structural stability, and thus can effectively increase the yield of carminic acid.

It will be apparent to those skilled in the art that various modifications and variations can be made to the disclosed embodiments. It is intended that the specification and examples be considered as exemplars only, with a true scope of the disclosure being indicated by the following claims and their equivalents.

What is claimed is:

1. A glycosyltransferase mutant, comprising:
at least one amino acid mutation relative to an amino acid sequence of a wild-type glycosyltransferase, wherein a site of the amino acid mutation is selected from a group consisting of sites 30, 33, 52, 92, 372, and any combination thereof, and wherein the site of the amino acid mutation is defined based on an amino acid sequence of SEQ ID NO: 1.

2. The glycosyltransferase mutant according to claim 1, wherein the site of the amino acid mutation is selected from a group consisting of: (1) substitution of G at site 30, (2) substitution of F at site 33, (3) substitution of I at site 52, (4) substitution of W at site 92, (5) substitution of Q at site 372, and (6) any combination of substitution modes of (1) to (5).

3. The glycosyltransferase mutant according to claim 2, wherein
G at site 30 is substituted by A.

4. The glycosyltransferase mutant according to claim 2, wherein
F at site 33 is substituted by L.

5. The glycosyltransferase mutant according to claim 2, wherein
I at site 52 is substituted by M.

6. The glycosyltransferase mutant according to claim 2, wherein
W at site 92 is substituted by one of the following amino acids: V, P, L, N, R, H, G, S, Y, D, K, A, E, Q, M, F, C and I.

7. The glycosyltransferase mutant according to claim 2, wherein
Q at site 372 is substituted by L.

8. The glycosyltransferase mutant according to claim 2, wherein
when W at site 92 is substituted by E, an enzymatic catalytic activity of the glycosyltransferase mutant is at least 4 times that of the wild-type glycosyltransferase.

9. The glycosyltransferase mutant according to claim 2, wherein
when G at site 30 is substituted by A, and F at site 33 is substituted by L, an enzymatic catalytic activity of the glycosyltransferase mutant is at least 3 times that of the wild-type glycosyltransferase.

10. The glycosyltransferase mutant according to claim 2, wherein
when G at site 30 is substituted by A, F at site 33 is substituted by L, and W at site 92 is substituted by E, an enzymatic catalytic activity of the glycosyltransferase mutant is at least 12 times that of the wild-type glycosyltransferase.

11. The glycosyltransferase mutant according to claim 2, wherein
when G at site 30 is substituted by A, F at site 33 is substituted by L, and W at site 92 is substituted by E, the glycosyltransferase mutant has a melting temperature at least 2° C. higher than that of the wild-type glycosyltransferase.

12. A method for catalyzing a glycosylation reaction, comprising
utilizing the glycosyltransferase mutant according to claim 1.

13. The method according to claim 12, wherein the glycosylation reaction is used for producing carminic acid.
